(19) United States
(12) Patent Application Publication
(10) Pub. No.: US 2025/0051665 A1
(43) Pub. Date: Feb. 13, 2025

(54) Process and System for Producing One or More Hydrocarbons

(71) Applicant: Linde GmbH, Pullach (DE)

(72) Inventors: Gunther Kracker, Weyarn (DE); Mathieu Zellhuber, Planegg (DE); Sean McCracken, Puchheim (DE); Andreas Meiswinkel, Rimsting (DE); Martin Schubert, München (DE); Desislava Tota, München (DE)

(21) Appl. No.: 18/717,874

(22) PCT Filed: Dec. 8, 2022
(86) PCT No.: PCT/EP2022/084975

§ 371 Date: Jun. 7, 2024

(30) Foreign Application Priority Data
Dec. 8, 2021 (EP) 21020622.3

(51) Int. Cl. C10G 69/06; C10G 9/36; C10G 27/00; C10G 29/04; C10G 45/62

(52) U.S. Cl. CPC C10G 69/06; C10G 9/36; C10G 27/00; C10G 29/04; C10G 45/62

(57) ABSTRACT

A method for producing one or more hydrocarbons, includes subjecting a first feed stream to a steam cracking to obtain a first product stream and subjecting a second feed stream containing ethane to an oxidative dehydrogenation to obtain a second product stream At least a portion of the first product stream is subjected to a treatment to obtain hydrocarbon fractions. The treatment includes a selective hydrogenation of hydrocarbons having two carbon atoms and a demethanization. At least a portion of the second product stream is subjected to a trace removal, which comprises the removal of oxygen and/or acetylene, to obtain an subsequent stream. At least a portion of the subsequent stream is fed to the treatment at a position downstream of the selective hydrogenation and upstream of the demethanization. A portion of the subsequent stream is subjected to a carbon dioxide removal upstream of the feed point into the treatment.

### CROSS-REFERENCE TO RELATED APPLICATIONS

This application is the national phase of, and claims priority to, International Application No. PCT/EP2022/084975, filed 8 Dec. 2022, which claims priority to European Patent Application No. EP21020622.3, filed 8 Dec. 2021.

### FIELD OF THE INVENTION

The invention relates to a method and a system for producing one or more hydrocarbons.

### BACKGROUND

Steam cracking of hydrocarbons is described, for example, in the article “Ethylene” in Ullmann's Encyclopedia of Industrial Chemistry, online edition, Apr. 15, 2009, DOI: 10.1002/14356007.a10_045.pub2. Steam cracking is used predominantly for obtaining short-chain olefins, such as ethylene and propylene, diolefins, such as butadiene, or aromatics, but is not limited thereto.

The oxidative dehydrogenation (ODH) of paraffins having two to four carbon atoms (also called ODHE or ODH-E in the case of the oxidative dehydrogenation of ethane to ethylene) is also known. During oxidative dehydrogenation, said paraffins are converted with oxygen, inter alia, to give the respective olefins and water. It represents an alternative, and in some cases advantageous, method for the production of olefins.

As explained below, combined methods of steam cracking and oxidative dehydrogenation are also known, for example from WO 2018/024650 A1, WO 2014/134703 A1 and CN 103086824 B.

### SUMMARY

An object of the invention is to improve corresponding combined methods.

According to an embodiment, a method for producing one or more hydrocarbons includes subjecting a first feed stream to a steam cracking to obtain a first product stream and subjecting a second feed stream containing ethane to an oxidative dehydrogenation to obtain a second product stream. At least a portion of the first product stream is subjected to a treatment to obtain hydrocarbon fractions (C2H4, C2H6), the treatment comprising the selective hydrogenation of hydrocarbons having two carbon atoms and a demethanization. At least a portion of the second product stream is subjected to a trace removal, which comprises the removal of oxygen and/or acetylene, to obtain an subsequent stream. At least a portion of the subsequent stream is fed to the treatment at a feed point downstream of the selective hydrogenation and upstream of the demethanization. At least a portion of the subsequent stream upstream of the feed point into the treatment is subjected to a carbon dioxide removal, wherein a residual oxygen content of the subsequent stream is less than 500 vol. ppm and wherein a residual acetylene content of the subsequent stream is less than 5 vol. ppm.

### BRIEF DESCRIPTION OF THE DRAWINGS

FIG. 1 illustrates a method in accordance with an embodiment of the invention.

FIG. 2 illustrates a method in accordance with another embodiment of the invention.

### WRITTEN DESCRIPTION

A method for producing one or more hydrocarbons is proposed, in which a first feed stream is subjected to steam cracking to obtain a first product stream and a second feed stream containing ethane is subjected to oxidative dehydrogenation to obtain a second product stream, wherein at least a portion of the first product stream is subjected to a treatment to obtain hydrocarbon fractions, the treatment comprising a selective hydrogenation of hydrocarbons having two carbon atoms and a demethanization, wherein at least a portion of the second product stream is subjected to a trace removal, which comprises the removal of oxygen and/or acetylene, to obtain a subsequent stream, and wherein at least a portion of the subsequent stream is fed to the treatment at a feed point downstream of the selective hydrogenation and upstream of the demethanization. At least a portion of the subsequent stream is subjected to a carbon dioxide removal upstream of the feed point into the treatment. Within the scope of the invention, this separate removal of carbon dioxide from the second product stream is particularly advantageous because it does not come at the expense of any capacity-limited removal downstream of the steam cracker.

A residual oxygen content of the subsequent stream in the proposed method is less than 500 vol. ppm and a residual acetylene content of the subsequent stream in the proposed method is less than 5 vol. ppm.

Furthermore, as explained below, this reduces or keeps the consumption of alkaline solution low, in particular when a fine cleaning of carbon dioxide from the first product stream is carried out using alkaline scrubbing. This is particularly the case because the first product stream, corresponding to its origin from the steam cracker, contains comparatively little carbon dioxide and therefore the removal of carbon dioxide from this first product stream can only be achieved in a technically and economically viable manner using an alkaline scrubbing. Upstream amine scrubbing is therefore not required and is typically not present in existing steam crackers. If such an alkaline scrubbing were also to be used to remove the larger amounts of carbon dioxide from the second product stream, a corresponding section of the system would have to be expanded considerably or a consumption of an alkaline solution would be high. This will certainly be prevented by the measures proposed here.

For the sake of clarification, it should be noted again that in all embodiments of the invention, at least a portion of the first product stream upstream of the feed point for the subsequent stream into the treatment is subjected to a carbon dioxide removal, which may in particular comprise an alkaline scrubbing, and in certain embodiments of the invention exclusively an alkaline scrubbing.

According to one embodiment of the invention, the removal of carbon dioxide from the subsequent stream or a portion thereof comprises at least one regenerative scrubbing, in particular an amine scrubbing and optionally fine cleaning by an alkaline scrubbing. In this way, a low residual carbon dioxide content can be achieved in a particularly advantageous manner. Such a low residual carbon dioxide content corresponds in particular, by its order of magnitude, to the residual content achieved in the carbon dioxide removal from the first product stream.

As also explained below, the invention provides an optimized integration of steam cracking and oxidative dehydrogenation, which allows the joint use of a demethanizer and a separation unit for the separation of hydrocarbons having two carbon atoms (C2 splitter). The trace removal makes it possible to avoid pressure-proofing or explosion-proofing system components. In particular, it is possible to optimize and adjust the ethylene capacity according to requirements.

According to one embodiment of the invention, oxidative dehydrogenation is carried out using one or more catalysts containing the metals molybdenum, vanadium, niobium and optionally tellurium. As explained below, corresponding catalyst systems are proven and robust and, in addition to advantageously obtaining olefins, also optionally allow the preparation of corresponding organic acids in co-production.

According to one embodiment of the invention, the treatment comprises a deethanization and/or a depropanization, wherein the selective hydrogenation is carried out downstream of the deethanization and/or downstream of the depropanization. The particular advantage of such an arrangement is that method steps, such as compression, alkali scrubbing and selective hydrogenation, in the separation section of a steam cracker system often limit capacity when retrofitting or expanding capacity. However, said method steps are advantageously not affected by an integration according to an embodiment of the invention. The method steps affected, in contrast, in particular separations, such as deethanization, demethanization and separation of hydrocarbons having two carbon atoms, often offer reserves or can be more easily expanded in their capacity, as recognized here.

According to one embodiment of the invention, the treatment comprises a deethanization, wherein selective hydrogenation is carried out upstream of the deethanization. Such a design particularly simplifies the subsequent separation of hydrocarbons having two carbon atoms and their subsequent separation from one another.

According to one embodiment of the invention, at least a portion of the subsequent stream is subjected (in addition to the removal of carbon dioxide) to a compression and/or a drying upstream of the feed point into the treatment. In this way, these potentially capacity-limiting steps in the treatment of the product mixture from steam cracking are not encumbered.

According to one embodiment of the invention, a catalyst containing at least palladium is used in the selective hydrogenation stage. Particularly advantageous and technically common catalysts are based on palladium, but can also be doped with other elements, in particular silver, gold, cerium, etc., in order to further improve the catalytic properties. Such catalysts are typically very sensitive to the carbon monoxide content in the process gas.

According to one embodiment of the invention, one or more catalysts containing copper oxide or one or more catalysts containing at least one of the elements copper, manganese, zinc, nickel, platinum, palladium, rhodium and/or ruthenium are used in the trace removal. Features and advantages are discussed, for example, in WO 2020/187572 A1, to which reference is therefore expressly made here.

According to one embodiment of the invention, a residual oxygen content of the subsequent stream is less than 250 vol. ppm, 100 vol. ppm, 10 vol. ppm or 1 vol. ppm. In particular, a higher oxygen content is possible than is typically the case in the raw-gas stream of a steam cracker since dienes are no longer present after in-feeding at the feed point and no fouling is expected. Nevertheless, the value is sufficiently low to avoid a safety hazard due to enrichment in a light fraction of methane and other low-boiling components, thus resulting in safety advantages. Because an extremely deep removal of oxygen is not required and because any remaining residual content is removed in a demethanization, in particular ethylene loss in a raw gas treatment is minimized. Details are explained below.

In general, the term “raw gas treatment” is understood to mean a treatment of the product stream from oxidative dehydrogenation, or a stream derived therefrom, regardless of the point at which it takes place in a corresponding process. However, raw gas treatment in the sense understood here always takes place before being combined with a stream from the steam cracking.

According to one embodiment of the invention, in the treatment downstream of the feed point, at least a portion of the gas mixture being processed in each case is cooled to temperatures below −120° C., −135° C. or −150° C. In particular, peak cooling can be used in demethanization, which results in a reduction of ethylene losses in the light fraction from the demethanization.

According to one embodiment of the invention, a residual acetylene content of the subsequent stream is less than 2 vol. ppm, 1 vol. ppm, 0.5 vol. ppm, 0.3 vol. ppm or 0.1 vol. ppm. On the one hand, corresponding values are particularly relevant to comply with the ethylene specification and, on the other hand, the raw gas treatment downstream of an oxidative dehydrogenation may produce particularly low acetylene contents.

According to one embodiment of the invention, the treatment comprises a separation of hydrocarbons having two carbon atoms in which an ethane-enriched stream is obtained, wherein the ethane-enriched stream is at least partially fed back to the steam cracking and/or oxidative dehydrogenation as part of the first and/or second feed stream. With such an embodiment, it is possible to expand the capacity of a steam cracker by using oxidative dehydrogenation, in particular for a corresponding ethane recycling.

According to one embodiment of the invention, traces are removed in a raw gas treatment, which is arranged upstream or downstream of a compression.

According to one embodiment of the invention, at least a portion of the second product stream is subjected to a condensate separation in which a condensate stream containing at least 1 wt. % acetic acid is separated, wherein the condensate stream is in particular subjected to further treatment in order to obtain acetic acid as a valuable product. In a corresponding embodiment, the invention enables an advantageous extraction of this valuable product.

A system which is designed to carry out a method in any embodiment of the invention is also the subject matter of the invention and benefits in the same way from the advantages mentioned above for individual embodiments and explained below.

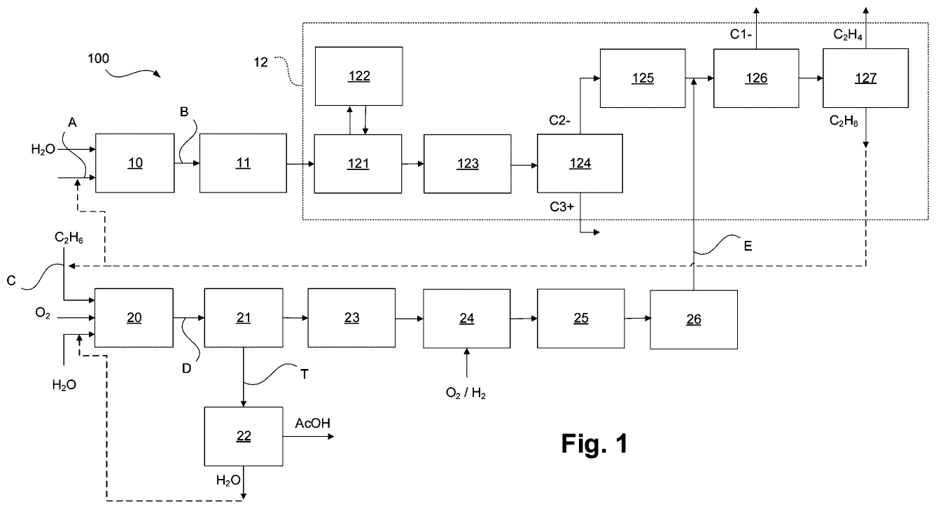

Turning now to FIG. 1, a method according to an embodiment of the invention is shown in the form of a schematic flowchart and is designated as a whole by 100.

The central method steps illustrated here are steam cracking 10 using one or more process units, such as cracking furnaces, and oxidative dehydrogenation 20, which can be carried out using one or more reactors. The steam cracking 10 is followed by a quenching step 11, which is customary in the art. This is followed by a treatment indicated overall by 12. The steps downstream of the oxidative dehydrogenation 20 are described below.

The treatment 12 comprises a compression 121, a carbon dioxide removal 122 (typically) undertaken at an intermediate stage of the compression 121, a drying 123, a deethanization 124 to obtain a heavier C3+ fraction containing hydrocarbons having three carbon atoms and heavier hydrocarbons and a lighter C2− fraction containing hydrocarbons having two carbon atoms, methane, carbon monoxide and optionally low-boiling components (such as residual oxygen), a selective hydrogenation 125 of hydrocarbons having two carbon atoms, a demethanization 126 to separate a light C1− fraction containing methane and other low-boiling components and a separation 127 of hydrocarbons having two carbon atoms from one another to obtain an ethylene fraction C₂H₄ and an ethane fraction C₂H₆. The latter can be fed back to the steam cracking 10 and/or oxidative dehydrogenation 20, as illustrated by a dashed arrow.

The oxidative dehydrogenation 20 is followed by a condensate separation 21 in which a condensate stream T is obtained. This is fed to a condensate treatment 22, in which an acetic acid fraction AcOH and a water fraction H₂O can be obtained. The latter can be fed back to the steam cracking 10 and/or oxidative dehydrogenation 20, as illustrated by a dashed arrow. The separation 21 of condensate is followed by a compression 23, downstream of which a trace removal 24 is carried out, which comprises removal of oxygen and/or acetylene, and to which oxygen O₂, carbon monoxide CO and/or hydrogen H₂ can optionally be supplied. This is followed by the removal 25 of carbon dioxide and an optional drying 26. Alternatively, a trace removal 24 can also be carried out upstream of the compression 23 or in an intermediate compression 23.

As illustrated here, a first feed stream, designated here as A, is fed to the steam cracking 10 and is processed there to obtain a first product stream, designated here as B. Water H₂O in the form of steam is also fed to the steam cracking 10. A second feed stream, designated here by C, containing ethane C₂H₆ is subjected to an oxidative dehydrogenation 20 to obtain a second product stream, designated here by D. Water H₂O in the form of steam and oxygen O₂ are also fed to the oxidative dehydrogenation 20. At least a portion of the first product stream B is subjected to a treatment 12, which comprises the aforementioned selective hydrogenation 125 of hydrocarbons having two carbon atoms and the likewise mentioned demethanization 126, to obtain (at least) the hydrocarbon fractions (C₂H₄, C₂H₆). At least a portion of the second product stream D (here the portion remaining after condensate separation) is subjected to (at least) the trace removal 24, which comprises the removal of oxygen and/or acetylene, to obtain a subsequent stream, designated here as E. At least a portion (here the remainder of the subsequent stream E remaining after carbon dioxide removal 25 and drying 26) is then fed to the treatment 12 already discussed above at a feed point downstream of the selective hydrogenation 125 and upstream of the demethanization 126. The selective hydrogenation 125 is carried out here as a front-end hydrogenation. The conducting of other material streams, which are not designated separately, is immediately apparent from the illustration. In embodiments of the invention, method steps can also be interchanged in an appropriate manner, e.g., in the form of a known “depropanizer first” process with subsequent selective hydrogenation.

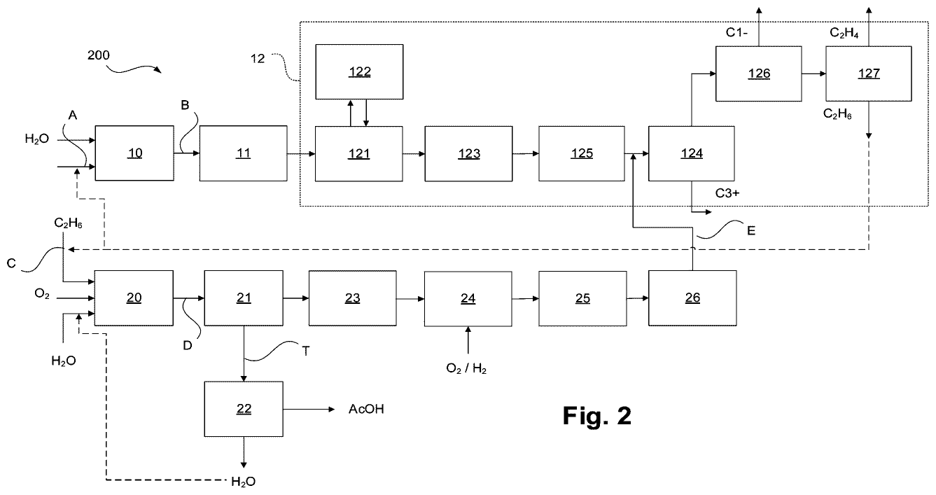

In FIG. 2, a method according to an embodiment of the invention is shown in the form of a schematic flowchart and is designated as a whole by 200. The embodiment 200 according to FIG. 2 differs from the embodiment 100 according to FIG. 1 substantially by the different selective removal 125, which is carried out here as raw gas hydrogenation. Reference is also made to the above observations.

The oxidative dehydrogenation of alkanes, in particular of ethane, may have advantages over more established methods for producing olefins, such as steam cracking or catalytic dehydrogenation. For instance, due to the exothermic nature of the reactions involved and the practically irreversible formation of water, there is no thermodynamic equilibrium limitation. Oxidative dehydrogenation can be carried out at comparatively low reaction temperatures. In principle, no regeneration of the catalysts used is required, since the presence of oxygen enables or causes regeneration in situ. Finally, in contrast to steam cracking, lower amounts of valueless by-products, such as coke, are formed. In embodiments of the invention, oxidative dehydrogenation, in particular of ethane, is particularly advantageous because it enables the advantageous use of ethane, even if steam cracking is designed to use (purely or predominantly) liquid feedstocks.

For further details regarding oxidative dehydrogenation, reference is made to the technical literature, for example: Ivars, F. and López Nieto, J. M., Light Alkanes Oxidation: Targets Reached and Current Challenges, in: Duprez, D. and Cavani, F. (ed.), Handbook of Advanced Methods and Processes in Oxidation Catalysis: From Laboratory to Industry, London 2014: Imperial College Press, pages 767-834, or Gartner, C. A. et al., Oxidative Dehydrogenation of Ethane: Common Principles and Mechanistic Aspects, ChemCatChem, Vol. 5, no. 12, 2013, pages 3196 to 3247, and X. Li, E. Iglesia, Kinetics and Mechanism of Ethane Oxidation to Acetic Acid on Catalysts Based on Mo—V—Nb Oxides, J. Phys. Chem. C, 2008, 125, 15001-15008.

Particularly relevant aspects of oxidative dehydrogenation in embodiments of the invention and further technical background of corresponding embodiments are explained in more detail below.

The product gas from the oxidative dehydrogenation of ethane downstream of a condensate separation contains significant amounts of acetylene, for example up to 500 vol. ppm, 400 vol. ppm, or 300 vol. ppm and more than 10 vol. ppm, 50 vol. ppm, or 100 vol. ppm, of carbon monoxide, for example up to 5 vol. %, 4 vol. %, or 3 vol. % and more than 0.5 vol. %, or 1 vol. %, of carbon dioxide, for example up to 4 vol. %, 3 vol. %, or 2 vol. % and more than 0.5 vol. % or 1 vol. % and of oxygen, for example up to 2 mol %, 1.5 mol %, 1 mol %, or 0.5 mol % and more than 500 vol. ppm, 1000 vol. ppm, or 2500 vol. ppm. Apart from acetylene, only trace amounts of other mono- and polyunsaturated hydrocarbons (higher olefins, dienes and higher acetylenes) are present in the product gas.

Corresponding options for removing residual oxygen and acetylenes, previously also referred to as “trace removal,” are known in principle and are disclosed, for example, in WO 2020/187572 A1 or in WO 2018/153831 A1. Typically, residual oxygen contents of less than 1000 vol. ppm, 500 vol. ppm, 100 vol. ppm, 10 vol. ppm, or 1 vol. ppm and acetylene contents of less than 5 vol. ppm, 2 vol. ppm, 1 vol. ppm, 0.5 vol. ppm, 0.3 vol. ppm, or 0.1 vol. ppm can be achieved. In embodiments of the invention, the complete removal of oxygen and/or acetylene is not necessary. The term “raw gas treatment” as defined above is also used below.

As described, for example, in WO 2018/153831 A1, oxygen, carbon monoxide and optionally acetylene can be removed from the product stream of an oxidative dehydrogenation of ethane. In this case, the use of an oxidation catalyst is generally disclosed, which may contain as components in particular the metals niobium, copper, zinc, palladium, silver, platinum, gold, iron, manganese, cerium, tin, rubidium and chromium. In this case, copper and/or platinum-based systems, but in particular copper-based systems, are preferably used. Embodiments of the invention can be carried out using any acetylene and oxygen removal process known from the prior art and the technical literature.

During oxidative dehydrogenation, in particular when using the catalyst systems mentioned above, acetic acid is formed as a co-product (typical ratio 3 to 12 mol/mol ethylene to acetic acid), but the demand for acetic acid is often limited to specific downstream products or processes (e.g., the production of vinyl acetate monomer, VAM). Acetic acid can be separated as a component of the condensate phase separated downstream of the oxidative dehydrogenation (the typical acetic acid content in the condensate is 1 to 30 wt. %, 3 to 25 wt. %, or 5 to 20 wt. %) and provided as a separate valuable product by suitable treatment, as explained.

On the other hand, in contrast to the steam cracker, apart from the by-products carbon monoxide and carbon dioxide, the oxidative dehydrogenation of ethane produces only ethylene and acetic acid as products. This also eliminates the requirement for appropriate recovery of other product fractions from the oxidative dehydrogenation and for corresponding equipment in the separation section. In embodiments of the invention, the use of corresponding products is particularly advantageous.

The technology used in steam cracking is also extensively regulated. What is typically important here is strict compliance with the product specification of ethylene with regard to downstream processes (e.g., polyethylene production, ethylene oxide production, etc.). Here, very strict limit values must be observed, particularly for carbon monoxide, carbon dioxide, oxygen and acetylene, which are achieved through appropriate cleaning and separation steps. Particularly relevant method steps are listed below and explained in more detail.

In embodiments of the invention, certain components of the product mixture or raw-gas stream in steam cracking and aspects of its further treatment are particularly relevant. In particular, a corresponding product mixture contains a high proportion of polyunsaturated hydrocarbons (dienes and acetylenes) and has a low carbon monoxide and carbon dioxide content. Aspects of treatment include the removal of carbon monoxide by demethanization (see below) and/or the removal of carbon dioxide, typically by alkali scrubbing (see below).

Selective hydrogenation of hydrocarbons having two carbon atoms serves to reduce the acetylene content according to specifications. Acetylene is selectively hydrogenated to ethylene using suitable catalysts. Selective hydrogenation used to be carried out predominantly with nickel-based catalysts, but nowadays mainly palladium-based catalysts with suitable dopants, such as silver, gold, cerium, etc., are used. There is considerable sensitivity to carbon monoxide. Carbon monoxide serves as a moderator and the carbon monoxide content in the hydrogenation is typically in the range of 50 to 2500 vol. ppm, 100 to 1500 vol. ppm, or 150 to 1000 vol. ppm. These carbon monoxide contents are significantly lower than the above-mentioned carbon monoxide contents of a process gas from an oxidative dehydrogenation of ethane and also lower than in cases where raw gas treatment is used.

High carbon monoxide levels in selective hydrogenation can be compensated for by a higher temperature, but this may lead to ethylene loss. The maximum temperature is limited on the one hand by the design temperature of the reactor, in particular in existing plants, but also by the operating window of the catalyst. If the above temperature range is exceeded, selective hydrogenation can no longer be carried out or the use of specially adapted catalysts is required.

In principle, selective hydrogenation is also not insensitive to the presence of oxygen, since water can be formed in the presence of oxygen, which in turn promotes the formation of other by-products, such as carboxylic acids and green oil. In embodiments of the present invention, corresponding problems are eliminated in a particularly advantageous manner.

Various variants are known as positions for selective hydrogenation:

(1) A raw-gas hydrogenation is positioned immediately downstream of the compression, alkali scrubbing and drying. This is preferably used in gas crackers, i.e., steam crackers operated with gaseous feedstocks. Higher polyunsaturated hydrocarbons are also converted, at least in part (in particular butadiene, methylacetylene and propadiene). Typical carbon monoxide levels are in the lower section of the above range, i.e., in particular in the range of 50 to 1000 vol. ppm or 50 to 500 vol. ppm.

(2) In a first embodiment, a so-called front-end hydrogenation is positioned downstream of a deethanization. This is therefore a pure acetylene hydrogenation and higher hydrocarbons than those with two carbon atoms are not contained in the feed stream of the selective hydrogenation. In both cases, in addition to ethane, corresponding amounts of methane, hydrogen and carbon monoxide are contained in the feed stream of the selective hydrogenation. The method results in typical carbon monoxide contents being higher here than in raw gas hydrogenation, i.e., in particular in the range of 100 to 2500 vol. ppm or 100 to 1500 vol. ppm. Another known variant is the arrangement downstream of a depropanization; in this case, the hydrogenation feed stream also comprises hydrocarbons having three carbon atoms. Similar to the hydrogenation of raw gas, the corresponding higher polyunsaturated hydrocarbons are then at least partially converted.

(3) In principle, a so-called tail-end hydrogenation is also known, in which at least one stream containing methane and other light components is separated upstream of the hydrogenation and accordingly stoichiometric hydrogen doping is required. However, this variant is less relevant in embodiments of the invention.

In embodiments of the invention, a raw-gas or front-end hydrogenation is used in particular, as explained in points (1) and (2).

In addition to the requirement for high product purity of the ethylene product, cryogenic system components also require the quantitative separation of carbon dioxide in order to avoid freezing of carbon dioxide and thus blockages.

Owing to its strong interaction with suitable solvents or washing liquids, carbon dioxide, in particular in contents that occur in the oxidative dehydrogenation of ethane, can likewise be removed comparatively easily from the product mixture, wherein it is possible to use known methods for removing carbon dioxide, in particular corresponding scrubbing (for example amine scrubbing). The laden scrubbing liquid is then regenerated in a separate column, and very pure carbon dioxide is released by desorption.

Should subsequent steps require the absence of, or only a very low residual concentration of, carbon dioxide (for example due to catalytic inhibition or so-called catalyst poisoning), the residual carbon dioxide content after amine scrubbing can be further reduced by an optional alkali scrubbing as fine cleaning, as required.

With certain exceptions, the corresponding scrubbing liquids can also react with oxygen, as a result of which disadvantageous aging or damage to the scrubbing agents, which require a continuous purge and makeup stream or lead to an undesired shortening of the service life of these scrubbing liquids, can occur over time. From this aspect as well, therefore, the removal of oxygen upstream of a corresponding scrubbing is advantageous.

The raw-gas stream from a steam cracker typically contains significantly lower carbon dioxide contents than in oxidative dehydrogenation. These are typically removed with an alkali scrubbing. A carbon dioxide content several orders of magnitude higher, as in the oxidative dehydrogenation of ethane, would lead to excessive alkali consumption, as previously stated. In the invention, as mentioned, a separate separation of carbon dioxide is therefore carried out by means of amine scrubbing and optionally alkali scrubbing downstream of the oxidative dehydrogenation.

Water is removed according to the prior art using regenerative dryers based on molecular sieves and, in addition to achieving the product specification, is also mandatory with regard to downstream cryogenic process steps in order to avoid blockages caused by the deposition of ice and hydrates.

Typically, as part of an appropriate process control, both in the oxidative dehydrogenation of ethane and in the separation section of a steam cracker, methane contained in the product stream (e.g., in particular from the ethane feed stream of the oxidative dehydrogenation) must also be removed. This typically involves demethanization, which requires appropriate cryogenic conditions. Demethanization simultaneously removes carbon monoxide and hydrogen from the corresponding stream. A fraction called C1− (pronounced “C1 minus”) is formed, which contains methane, hydrogen and/or carbon monoxide as substantial components. Traces of oxygen still contained in the inlet stream of the demethanization also enter this fraction. Typically therefore the aim is to limit the oxygen content in the inlet stream to the demethanization and thus avoid the possible formation of an explosive atmosphere at the top. A corresponding design of the demethanizer is possible, as described for example in WO 2018/082945 A1, but this means significantly increased equipment expenditure. EP 3 456 703 A1 describes a demethanization in the separation section of a system for the oxidative dehydrogenation of ethane, which is combined with a pressure swing adsorption in the overhead stream. To minimize product losses, the aim is also to minimize the ethylene content in the top gas of the demethanizer.

Ultimately, unreacted ethane has to be separated from ethylene, which takes place by means of a C2 splitter, which is likewise operated under cryogenic conditions. It must therefore be constructed and operated in such a way that ethane in the ethylene is practically quantitatively removed (required purity of the ethylene product is usually more than 99.9%) and, at the same time, an ethane stream that is fed back for oxidative dehydrogenation contains as little ethylene as possible or no ethylene.

Approaches to integrating steam cracking and oxidative dehydrogenation of ethane are basically known, as already mentioned. What all of the above-mentioned documents have in common is that they do not address the well known formation of acetic acid as a co-product of oxidative dehydrogenation and in each case only separate an aqueous condensate phase. Embodiments of the invention, however, also take this aspect in particular into consideration.

WO 2018/024650 A1 focuses in particular on the integration of a steam cracker with an ethane feed and with an oxidative dehydrogenation of ethane. The feeding of a process gas from an oxidative dehydrogenation of ethane into the separation section of a steam cracker is described and claimed in principle. However, no solutions have been disclosed which address and solve the problem of the significantly different oxygen, carbon monoxide and acetylene contents in the process gas from an oxidative dehydrogenation of ethane and in corresponding streams in the separation section of a steam cracker. A tail-end hydrogenation and a merging of product streams upstream of this tail-end hydrogenation are described, so that the hydrocarbons having two carbon atoms from steam cracking and the oxidative dehydrogenation of ethane are subjected to a joint hydrogenation. This is in direct contrast to the solution of the embodiments of the invention. Accordingly, raw gas treatment of the process stream of an oxidative dehydrogenation of ethane is neither shown nor claimed.

WO 2014/134703 A1 discloses an oxygen removal immediately downstream of a reactor for the oxidative dehydrogenation of ethane (so-called “afterburner”), with typical residual oxygen contents of less than 1000 vol. ppm being given. In general, approaches to integrate the oxidative dehydrogenation of ethane and steam cracking are claimed, which optionally include the process units C2 splitter and/or acetylene hydrogenation. The integration can take place both explicitly upstream and explicitly downstream of the acetylene hydrogenation. However, solutions to the problem of the significantly different carbon monoxide and acetylene contents in the process gas from an oxidative dehydrogenation of ethane and in corresponding streams in the separation section of a steam cracker are not presented here. The advantageous positioning of a demethanizer and integration in the overall process is also not discussed in detail in the document.

CN 103086821 B relates to the integration of a naphtha cracker with oxidative dehydrogenation of ethane. While it does provide basic propositions for the removal of oxygen, carbon monoxide and carbon dioxide, the document does not disclose a solution for the removal of acetylene. Rather, in contrast to the previous statements, this document teaches that the oxidative dehydrogenation of ethane should not produce by-products, such as acetic acid and acetylene.

Embodiments of the invention meet the contrasting requirements outlined above and enable optimized integration of an oxidative dehydrogenation of ethane and a steam cracker.

A number of aspects prevent the product gas from being fed from the oxidative dehydrogenation of ethane into the separation section of a steam cracker. For example, a high carbon monoxide content in the process gas of the oxidative dehydrogenation of ethane is limiting for the hydrogenation of hydrocarbons having two carbon atoms; here, only a so-called tail-end hydrogenation can be used initially or only special catalysts that tolerate high carbon monoxide content can be used. Too high a carbon dioxide content in the process gas the carbon monoxide content goes against removal by means of typical alkali scrubbing. The oxygen content in the process gas from the oxidative dehydrogenation can lead to fouling effects, a possible enrichment of oxygen in the mentioned C1 minus fraction, and therefore to a safety hazard. Even after a separate raw gas treatment in the process gas of the oxidative dehydrogenation, the polyunsaturated hydrocarbons from the cracker mean that the oxygen content can still be critical, or there is a very strict purity requirement (less than 10 vol. ppm, in particular less than 1 vol. ppm oxygen) for such a raw gas treatment, which requires corresponding effort and can lead to ethylene losses.

Embodiments of the invention solve these problems by the repeatedly mentioned feeding of the product gas stream from an oxidative dehydrogenation, after suitable pretreatment at a suitable point, into the separation section of a steam cracker. On the one hand, in embodiments of the invention, the pretreatment of the product gas stream of the oxidative dehydrogenation comprises a separate compression and raw gas treatment and, optionally, carbon dioxide removal (in particular amine scrubbing). Here carbon dioxide is advantageously obtained as a separate pure product for possible further use and/or so-called carbon capture & storage (CCS), for example in the form of injection into the ground. Preferably, the carbon dioxide is removed from the product gas stream of the oxidative dehydrogenation of ethane in two stages (amine scrubbing and alkali scrubbing) in order to achieve similarly low carbon dioxide contents to those in the cracked gas of the cracker downstream of the alkali scrubbing (usually values less than 1 mol-ppm or even lower) before being fed into the separation section of a steam cracker. If necessary, drying takes place. On the other hand, in embodiments of the invention, feeding into the separation section of a steam cracker takes place downstream of a selective hydrogenation of hydrocarbons having two carbon atoms (front-end or raw-gas hydrogenation) and upstream of a demethanization.

Necessary adjustments to the temperature levels are not explicitly taken into account here or in FIGS. 1 and 2 explained above and are carried out in a manner known to a person skilled in the art (e.g., heat exchanger).

In one embodiment of the invention, at least portions of the combined product gas stream are particularly preferably cooled at individual points in the process to temperatures of less than −120° C., −135° C., or −150° C. Reaching particularly low temperatures promotes the minimization of ethylene losses in the light C1− fraction, which, as mentioned, is separated from the combined product gas stream in the demethanizer.

This results in an additional synergy advantage of the integration, since only a small C1− fraction is available in a stand-alone, non-integrated system for the oxidative dehydrogenation of ethane. This is not suitable for the generation of usable peak cooling and temperatures below −110° C. cannot be achieved in a stand-alone oxidative dehydrogenation system without considerable additional effort. This situation improves significantly if the product gas stream from the oxidative dehydrogenation of ethane is combined with that from the cracker and fed to a common demethanizer. Thus, ethylene losses can be reduced at least partially for the fraction from the oxidative dehydrogenation of ethane by the availability of peak cooling in the above-mentioned temperature range.

1. A method for producing one or more hydrocarbons,
comprising:
subjecting a first feed stream to a steam cracking to obtain a first product stream;
subjecting a second feed stream containing ethane to an oxidative dehydrogenation to obtain a second product stream;
subjecting at least a portion of the first product stream to a treatment to obtain hydrocarbon fractions, the treatment comprising the selective hydrogenation of hydrocarbons having two carbon atoms and a demethanization;
subjecting at least a portion of the second product stream to a trace removal, which comprises the removal of oxygen and/or acetylene, to obtain an subsequent stream;
feeding at least a portion of the subsequent stream to the treatment at a feed point downstream of the selective hydrogenation and upstream of the demethanization; wherein
subjecting at least a portion of the subsequent stream upstream of the feed point into the treatment to a carbon dioxide removal, wherein a residual oxygen content of the subsequent stream is less than 500 vol. ppm and wherein a residual acetylene content of the subsequent stream is less than 5 vol. ppm.

2. The method according to claim 1, wherein the removal of carbon dioxide comprises at least one regenerative scrubbing, in particular an amine scrubbing.

3. The method according to claim 2, wherein the oxidative dehydrogenation is carried out using one or more catalysts containing the metals molybdenum, vanadium, and niobium.

4. The method according to claim 1, wherein the treatment comprises a deethanization and/or a depropanization, and wherein the selective hydrogenation is carried out downstream of the deethanization and/or downstream of the depropanization.

5. The method according to claim 1, wherein the treatment comprises a deethanization, and wherein the selective hydrogenation is carried out upstream of the deethanization.

6. The method according to one claim 1, further comprising:
subjecting at least a portion of the subsequent stream to compression and/or drying upstream of the feed point into the treatment.

7. The method according to claim 1, wherein a catalyst containing at least palladium is used in the selective hydrogenation.

8. The method according to claim 1, wherein one or more catalysts containing copper oxide or one or more catalysts containing at least one element selected from the group consisting of copper, manganese, zinc, nickel, platinum, palladium, rhodium and ruthenium are used in the trace removal.

9. The method according to claim 1, wherein a residual oxygen content of the subsequent stream is less than 250 vol. ppm.

10. The method according to claim 1, wherein a residual acetylene content of the subsequent stream is less than 2 vol. ppm.

11. The method according to claim 1, wherein the treatment comprises a separation of hydrocarbons having two carbon atoms in which an ethane-enriched stream is obtained, and wherein the ethane-enriched stream is at least partially fed back to the steam cracking and/or oxidative dehydrogenation as part of the first and/or second feed stream.

12. The method according to claim 1, wherein the trace removal is carried out by means of a raw gas treatment which is arranged upstream or downstream of a compression.

13. The method according to claim 1, wherein, in the treatment downstream of the feed point, at least a portion of the gas mixture being processed in each case is cooled to temperatures below −120° C., 135° C. or 150° c.

14. The method according to claim 1, wherein at least a portion of the second product stream is subjected to a separation of condensate, in which a condensate stream is separated, which contains at least 1 wt. % acetic acid, wherein the condensate stream is in particular subjected to further treatment in order to obtain acetic acid as a valuable product.

15. (canceled)

16. The method according to claim 1, wherein the oxidative dehydrogenation is carried out using one or more catalysts containing the metals molybdenum, vanadium, and niobium.

17. The method according to claim 16, wherein the one or more catalysts further contain the metal tellurium.

18. The method according to claim 3, wherein the one or more catalysts further contain the metal tellurium.

19. The method according to claim 2, wherein the treatment comprises a deethanization, and wherein the selective hydrogenation is carried out upstream of the deethanization.

20. The method according to claim 9, wherein the residual oxygen content of the subsequent stream is less than 100 vol. ppm.

21. The method according to claim 10, wherein the residual acetylene content of the subsequent stream is less than 1 vol. ppm.
